(19) United States
(12) Patent Application Publication
(10) Pub. No.: US 2003/0044862 A1
(43) Pub. Date: Mar. 6, 2003

(54) Diagnostic marker for tumor hypoxia and prognosis

(75) Inventors: Amato J. Giaccia, Stanford, CA (US); Quynh Thu Le, San Carlos, CA (US); Nicholas C. Denko, Menlo Park, CA (US)

Correspondence Address: SPECKMAN LAW GROUP, 1501 WESTERN AVE, SUITE 100, SEATTLE, WA, 98101, US

(73) Assignee: THE BOARD OF TRUSTEES OF THE LELAND STANFORD JR. UNIVERSITY, Palo Alto, CA

(21) Appl. No.: 10/175,440

(22) Filed: Jun. 18, 2002

Related U.S. Application Data
(60) Provisional application No. 60/300,562, filed on Jun. 22, 2001.

(51) Int. Cl. G01N 33/574; A61K 31/00

(52) U.S. Cl. 435/7.23; 514/1

(57) ABSTRACT

Osteopontin (OPN) levels measured in bodily fluids of cancer patients are used as a noninvasive marker for tumor hypoxia as described herein. Methods of using OPN levels for the diagnosis, prognosis and treatment of cancers characterized by the presence of hypoxic cells, and a kit for detecting the presence of a hypoxic tumor are provided.

### CROSS REFERENCE TO RELATED APPLICATIONS

This application claims priority to U.S. Provisional Patent Application No. 60/300,562 filed Jun. 22, 2001.

### GOVERNMENT INTEREST

This invention was made with Government support under PHS Grant No. CA67166 awarded by the National Cancer Institute. The Government may have certain rights in the invention.

### FIELD OF THE INVENTION

The present invention relates to the noninvasive diagnosis of tumor hypoxia based on the measurement of osteopontin levels in the bodily fluids of cancer patients. Analysis of osteopontin levels in bodily fluids is also useful for guiding the design of hypoxia-specific cancer therapy and for predicting risk of relapse after therapy.

### BACKGROUND

Osteopontin

Osteopontin is a secreted calcium glycophosphoprotein that is expressed in normal tissues, such as bone, kidney, smooth muscle cells, endothelia, activated T cells and macrophages (1), and is associated with various tumors. High levels of osteopontin are found in the plasma of patients with metastatic breast cancer (2) and ovarian tumors (32). Osteopontin is expressed in a high percentage of premalignant and malignant oral lesions, but not normal oral epithelium (3). Increased tissue expression is correlated with tumor progression in gastric carcinomas (4), human gliomas (5), and colorectal cancers (33).

Tumor Hypoxia

The microenvironment of malignantly transformed cells in solid tumors affects both the malignant progression of transformed cells as well as their response to therapy. As a result of abnormal vasculature, many solid tumors possess poorly perfused regions that exist at oxygen tensions substantially below that of normal tissues. Some tumor cells become hypoxic and eventually anoxic due to limitations in oxygen diffusion from blood vessels to the expanding tumor mass (6). Others become transiently hypoxic through the temporary occlusion of blood flow (7). Tumor hypoxia has direct effects on therapeutic outcome. Hypoxic cells exhibit increased resistance to radiotherapy due to decreased levels of oxygen, are exposed to lowered levels of chemotherapeutic agents because of impaired delivery, and, as proliferation declines, become increasingly refractory to antiproliferative chemotherapy. Tumor hypoxia can also promote tumor progression, by selecting tumor cell variants with diminished apoptotic potential, stimulating pro-angiogenic gene expression and increasing metastastic potential (7). Hypoxia produces changes in gene transcription that may result in a more aggressive phenotype, e.g., genes involved in tissue remodeling and invasion (8,9).

Clinical studies have also indicated that hypoxia increases tumor invasiveness and dissemination. Tumor hypoxia predicts for a higher rate of distant metastasis in patients with soft-tissue sarcomas (10). Similarly, there is reportedly a higher risk of distant tumor spread or tumor relapse in patients with hypoxic cervical cancers compared to those with aerobic tumors (11,12). In squamous cell carcinoma of the head and neck (HNSCC), hypoxia is a major contributing factor to tumor recurrence. There is a strong correlation between pretreatment tumor oxygen status as measured by microelectrode measurements and tumor control and survival in HNSCC patients treated with radiotherapy (13, 14).

The availability of a molecular marker whose presence in a body fluid correlates highly with tumor hypoxia would have considerable clinical utility in the diagnosis, treatment and prognosis of cancer. At the present time, the most widely accepted method for measuring tumor hypoxia in patients is with the Eppendorf pO₂ polarographic microelectrode (15). However, its use involves an invasive procedure that requires direct insertion of a needle through multiple tracts of a tumor to obtain multiple samplings of tumor oxygen tension. Microelectrodes are cumbersome to manipulate, require a high level of patient cooperation, and are difficult to use routinely in deeply seated tumors. In addition, the equipment is costly to maintain, requires technical expertise to operate and is available only in a limited number of institutions.

### SUMMARY OF THE INVENTION

The present invention relates to the use of osteopontin (OPN) as a noninvasive marker for tumor hypoxia in cancer patients. As disclosed herein, OPN levels are elevated in bodily fluids of patients with hypoxic tumors and measurement of these levels can be carried out conveniently for the purpose of diagnosis, prognosis and therapy.

In one of its aspects, the invention provides a method of diagnosing tumor hypoxia by detecting a level of OPN in a bodily fluid of a patient with cancer and comparing the level with a predetermined value or values.

In another of its aspects, the invention provides a method of treating a patient with a malignant tumor, comprising diagnosing whether the tumor is hypoxic as described above, and administering a hypoxia-selective tumor therapy if the presence of a hypoxic tumor is diagnosed.

In yet another aspect, the invention provides a method for modulating, preferably enhancing, the response of a tumor to radiation or chemotherapy, comprising diagnosing whether the tumor is hypoxic as described above, and administering a hypoxic sensitizing agent combined with the radiation or chemotherapy treatment.

In still another aspect, the invention provides a method of monitoring the response of a patient to tumor therapy, preferably including hypoxia-selective tumor therapy, comprising determining a level of OPN in a bodily fluid obtained from the patient prior to therapy, and at various times during and after therapy, and comparing the level to a predetermined value or values that are indicative of the presence of tumor hypoxia.

The invention also provides a kit containing at least one binding agent for detecting OPN and calibration means for comparing the level of OPN with a predetermined value or values corresponding to tumor hypoxia. Examples of suitable calibration means include standardized OPN in amounts corresponding to a predetermined value or range of values, instructions for using the kit, a description of OPN levels in various bodily fluids corresponding to negative and positive results for tumor hypoxia, and others.

### BRIEF DESCRIPTION OF THE FIGURES

FIG. 1: VHL versus OPN Expression in cell lines. The values shown on the OPN and VHL axes represent the log₂ absolute expression values for OPN and VHL, respectively.

FIG. 2: Comparison of mean plasma OPN levels in VHL patients and healthy volunteers by ELISA assay.

FIG. 3: ELISA analysis of OPN expression in cell lysates and serum-free conditioned media from SCC4 cells after various times of hypoxia exposure (Hyp) or 24 hours of normoxia (Nor).

FIG. 4: Correlation between plasma OPN levels and medium tumor pO₂ in patients with HNSCC.

FIG. 5: Kaplan-Meier estimates of freedom from relapse and overall survival in relation to OPN plasma levels. A: freedom from relapse in patient study population; B: overall survival in patient study population; C: overall survival in patients with N0-2 neck nodes; D: overall survival in patients with N3 neck nodes.

### DETAILED DESCRIPTION OF THE PREFERRED EMBODIMENTS

The preferred embodiments of this invention are based on the inventors' studies, summarized below, which led to the unexpected identification of secreted osteopontin (OPN) as a clinical marker for tumor hypoxia.

In order to identify genes in cancer cells whose expression is susceptible to modulation by hypoxia, the NCI-60 cancer cell line microarray expression database was analyzed for changes in mRNA levels in cells with high or low expression of the von Hippel-Lindau tumor suppressor gene (VHL). The VHL tumor suppressor gene has been shown to modulate hypoxia-induced gene expression. VHL is a dominantly inherited genetic condition associated with the development of hemangioblastoma, renal cell carcinoma and pheochromocytoma (16). The VHL protein is part of a multi-protein complex that regulates the oxygen-dependent ubiquitination and proteolysis of the Hypoxia-Induced Factor-1 transcription factor (HIF-1) (17). The loss of VHL function substantially decreases HIF-1 degradation under aerobic conditions, and increases the expression of downstream genes such as endothelin 1, differentiated embryo chondrocyte 1, transglutaminase 2 and low density lipoprotein receptor-related protein 1 (18).

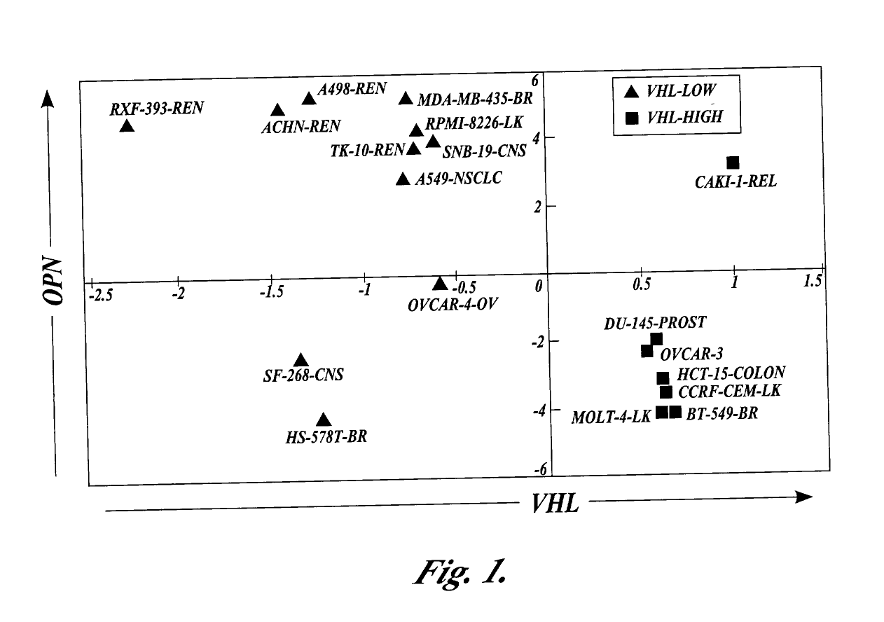

Linear Discriminant Analysis (LDA) (19, 20) was used to distinguish genes with high VHL expression from those with low VHL expression (FIG. 1; Example 1 below). This analysis showed OPN to be the highest-weighted gene in the profile, and the most predictive of VHL status in the dataset. Its expression inversely correlated with VHL expression.

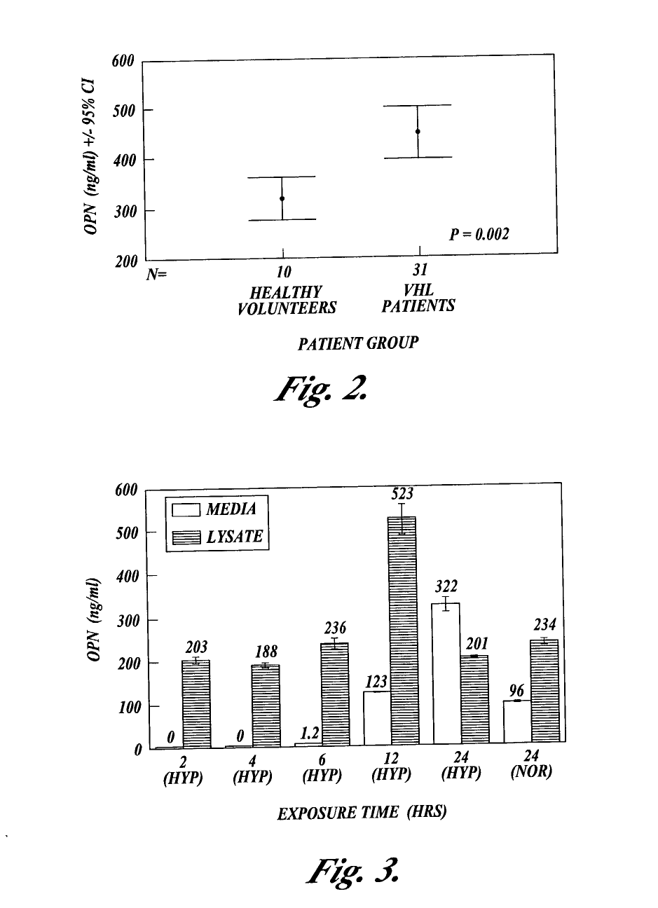

The plasma levels of OPN in VHL patients with a VHL mutation were compared with those of healthy volunteers to determine whether there was a relationship between VHL and OPN in human patients. These studies found a statistically significant difference in OPN levels between the two groups (FIG. 2; Example 2 below).

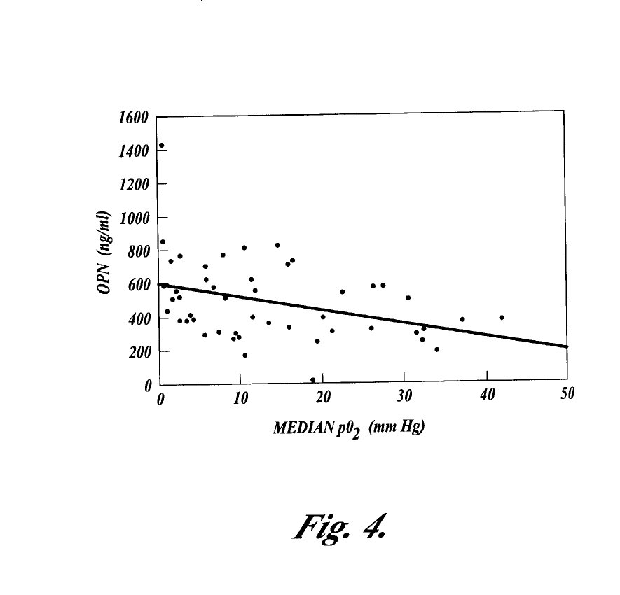

To assess whether OPN is regulated by hypoxia, the levels of intracellular and secreted OPN proteins were assessed by exposing SCC4, a HNSCC cancer cell line, to normoxic or hypoxic conditions and measuring OPN levels in lysates and media over a 24 hour period. Normoxic controls showed a low level of OPN secretion in the media after 24 hours, whereas OPN levels in the media increased progressively with time of hypoxia exposure (FIG. 3; Example 5).

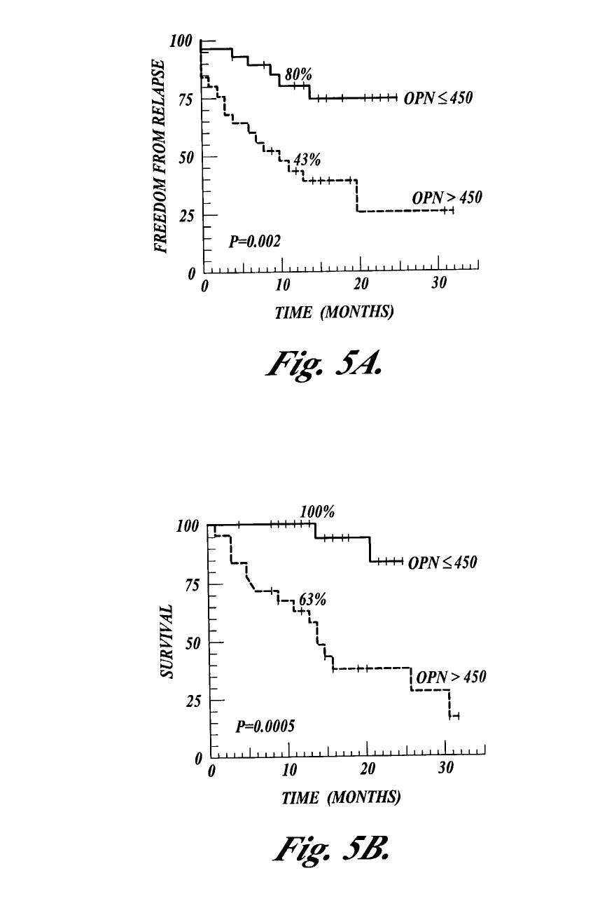

The relationship between secreted OPN levels and tumor hypoxia was assessed by measuring plasma OPN levels and tumor pO₂ in 49 patients with previously untreated squamous cell carcinoma of the head and neck (HNSCC). Measurement of tumor oxygenation was carried out using the Eppendorf microelectrode method. A significant correlation was found between OPN levels and tumor pO₂ (FIG. 4; Example 3).

Plasma levels of secreted proteins encoded by genes whose expression is increased in response to hypoxia, e.g., PAI-1, uPA, uPAR, TF and VEGF (8, 9, 21), were measured in healthy volunteers and 28 of the 49 head and neck cancer patients (Table 1). There was a trend for higher plasma levels of PAI-1, TF and VEGF in cancer patients compared to healthy volunteers, but there was no significant difference in the levels for patients with hypoxic tumors compared with aerobic tumors.

TABLE 1
Mean plasma levels of PAI-1, uPA, uPAR, TF, VEGF and OPN by tumor
pO₂

| | Healthy | | |
|---|---|---|---|
| Factor | Volunteers | Aerobic Tumors | Hypoxic Tumors |
| Mean PAI-1 | 22.1 | 31.9 | 35.5 |
| (range, ng/ml) | (5.4-36.5) | (9.3-56.4) | (12.3-69.9) |
| Mean UPA | 0.8 | 0.8 | 0.7 |
| (range, ng/ml) | (0.5-1.3) | (0.5-2.5) | (0.5-1.1) |
| Mean UPAR | 0.8 | 2.5 | 1.0 |
| (range, ng/ml) | (0.4-1.3) | (0.4-13.5) | (0.6-1.9) |
| Mean TF | 0 | 22.8 | 60.6 |
| (range, pg/ml) | (0-0) | (0-78.3) | (0-413.8) |
| Mean VEGF | 27.8 | 147.5 | 202.8 |
| (range, pg/ml) | (2-108) | (12.0-391.0) | (16.0-799.0) |
| Mean OPN | 303.0 | 373.8 | 600.9 |
| (range, ng/ml) | (258.9-411.7) | (22.2-730.1) | (227.2-1434.6) |

Studies were undertaken to identify potential predictors for tumor hypoxia. Of the variables evaluated, which included patient age, gender, pack-year of cigarette use, tumor stage, nodal stage, tumor volume, hemoglobin and OPN, only OPN levels predicted for a hypoxic tumor. Multivariate analysis by forward stepwise linear regression showed that OPN was the only significant independent predictor for tumor pO₂ (p=0.01, R=−0.42).

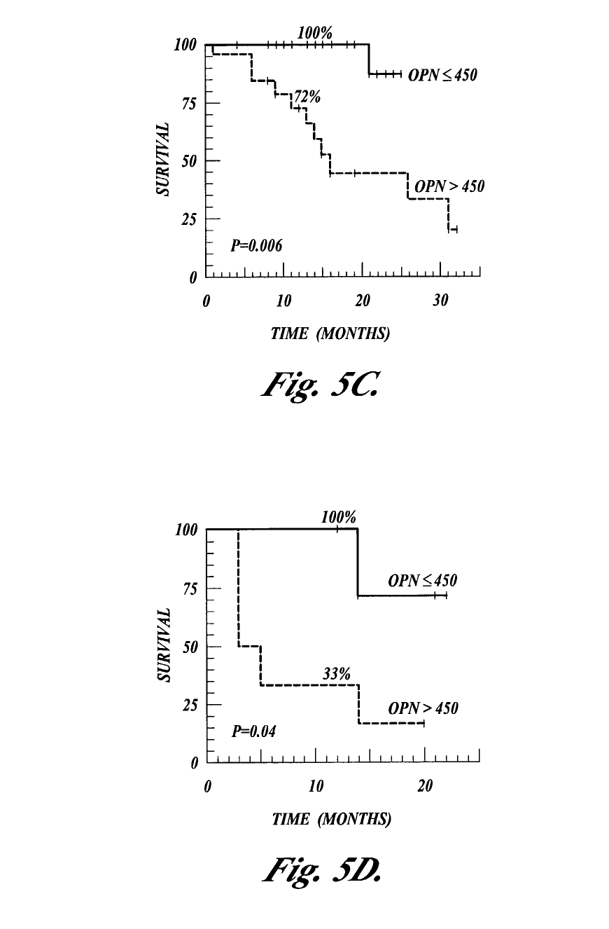

In addition to being a useful molecular marker for tumor hypoxia, OPN levels can be used prognostically to assess the likelihood of relapse after therapy. Analysis of the disease-free period in patients with HNSCC showed that patients in the high OPN group (above the group median, OPN>450 ng/ml) had significantly poorer tumor control than those with low OPN (below the group median, OPN≦450 ng/ml). The one year freedom from relapse (FFR) rate was 80% for patients with OPN levels less than or equal to 450 ng/ml and 43% for patients with OPN levels greater than 450 ng/ml (FIG. 5A). OPN was an important prognostic factor for survival (P=0.0005, FIG. 5B). The 1-year overall survival was 82%. The 1- year survival rate was 100% for patients with OPN levels less than or equal to 450 ng/ml and 63% for patients with OPN levels greater than 450 ng/ml.

Patients with hypoxic tumors (median pO₂≦10mm Hg) also showed poorer tumor control than those with aerobic tumors (median pO₂>10 mm Hg). The one and two-year FFR rates were 64% and 50% respectively for patients with median pO₂≦10 mm Hg, and were 71% and 67% respectively for patients with median pO₂>10 mm Hg. Although patients with hypoxic tumors had lower FFR rates than those with more oxic tumors, tumor pO₂ was not an independent predictor for treatment outcomes in this study, possibly owing to the fact that seventeen patients received Tirapzamine(TPZ) in combination with standard chemoradiotherapy as part of an institutional phase II study. The use of TPZ, a hypoxic cell toxin, may have eliminated hypoxic cells thereby discounting the adverse effect of tumor hypoxia in this group of patients.

Multivariate analysis showed plasma OPN level was an independent predictive factor for both FFR (p=0.007, hazard ratio: 3.7) and survival (p=0.02, hazard ratio: 12.6) (Table 2). It was the most significant predictor for both tumor relapse and survival, after adjusting for the other factors significant in univariant analysis (i.e., age and the use of chemotherapy). Univariate analysis revealed that age, N-stage, tumor volume and OPN levels were potential prognostic factors for survival with p-values<0.05. Multivariate analysis including only these four significant factors showed that OPN levels (favoring low levels, p=0.01) and N-stage (favoring N 0-2 patients, p=0.005) were significant predictors for survival. Age was of borderline significance (p=0.06). Within each N-stage group (N 0-2 and N3), OPN also appeared to be an independent predictor for survival (FIGS. 5C and 5D).

TABLE 2
Multivariate Cox proportional hazard model analysis

| | Freedom from Relapse | | | Overall Survival | | |
|---|---|---|---|---|---|---|
| Parameter | P | HR | 95% CI | P | HR | 95% CI |
| Age | 0.12 | 1.03/yr | 0.99-1.06 | 0.01 | 1.05/yr | 1.01-1.09 |
| (continuous) | | | | | | |
| OPN | 0.007 | 3.7 | 1.24-8.66 | 0.02 | 12.6 | 1.6-98.5 |
| (≦450 vs. > | | | | | | |
| 450 ug/ml) | | | | | | |
| Chemotherapy | 0.12 | 0.47 | 0.2-1.3 | | | |
| (yes vs. no) | | | | | | |
| TV (≦28 vs. > | | | | 0.71 | 1.2 | 0.4-4.2 |
| 28 cm³) | | | | | | |
| N-stage | | | | 0.005 | 6.2 | 1.8-22.1 |
| (0-2 vs. 3) | | | | | | |

HR: Hazard ratio; CI: confidence interval

The present invention provides a noninvasive method for assessing tumor hypoxia in cancer patients and for identifying patients at high risk for tumor recurrence.

In one of its aspects, the present invention provides a method of determining the presence or absence of tumor hypoxia in a patient with cancer comprising detecting a level of osteopontin in a bodily fluid obtained from the patient and comparing the level with a predetermined value.

“Osteopontin” or “OPN” refers to a secreted highly acidic glycophosphoprotein characterized by a conserved GRGDS amino acid sequence that includes, without limitation, the proteins described in references 1 (and references cited therein) and 22, and homologous proteins. Osteopontin is also known as ETA-1, bone sialoprotein I, 44 kDa-bone phosphoprotein, uroprotein, major transformation phosphoprotein, and activation protein-1. As used herein, the term encompasses variants and fragments of OPN, i.e., naturally occurring forms of OPN that are substantially similar but nonidentical to described OPN in sequence and/or length and are capable of substituting for OPN in a specific binding interaction, as described below.

Insofar as OPN is secreted into bodily fluids, e.g., blood (plasma, serum), lymph, bile, milk, saliva, tears and others, the level of OPN in a patient can be detected and monitored using noninvasive procedures. A level of OPN in a bodily fluid, preferably blood, may be detected by any protein chemistry analytical techniques or bioassays capable of identifying and quantitating OPN. These methods are well-known in the art and are routinely used by those of ordinary skill in protein analysis and/or bioassay methodology. A preferred method involves the use of a binding agent that reacts with OPN (or with a variant or fragment thereof) in a highly selective manner. The binding agent itself may contain a reporter group (e.g., a radioisotope, a fluorescent compound, a fluorescence emitting metal of the lanthanide series, a chemiluminescent or phosphorescent molecule, a paramagnetic group, or an enzyme). Alternatively, a detecting reagent containing a suitable reporter molecule, and capable of binding the binding agent-OPN complex, may be used (e.g., an anti-immunoglobulin, protein A, protein G). Examples of useful binding agents include antibodies, receptors, ligands or carrier molecules. Preferably, detection is carried out using immunoassay methods and immunoreagents that are well known in the art (see, e.g., ref. 26).

The term “antibody” is intended to refer to intact antibody molecules and antigen-binding fragments such as Fab and F(ab′)₂ that are produced by proteolytic cleavage of intact antibodies. Monoclonal antibodies or polyclonal antibodies that are directed against one or more epitopes of OPN can be used in the methods described herein. Polyclonal antibodies to OPN can be obtained from the sera of animals that are immunized with OPN or from commercial sources. Monoclonal antibodies can be prepared by methods known to those skilled in the art (see, e.g., ref. 27).

Immunoassays are carried out in solution or, preferably, on a solid phase support that is capable of binding antigen or antibody. In a “two-antibody sandwich type” immunoassay, a purified antibody is bound to a solid support and the support is contacted with the test fluid sample for sufficient time to allow the antigen in the sample (i.e., OPN) to bind to the antibody. After washing to remove unbound proteins, a second antibody is allowed to bind to the antigen. This antibody is labeled with a reporter group and is directed against an epitope on the antigen that differs from and is nonoverlapping with the epitope bound by the immobilized antibody. After washing, the amount of labeled second antibody bound to the solid support is measured. Either monoclonal antibodies or affinity-purified polyclonal antibodies can be used for this assay. The detection limit of the assay is typically about 0.01-0.1 ng antigen. The sensitivity of the assay can be varied by choice of a suitable label.

A preferred assay for use in the practice of this invention is a “two-antibody sandwich type” assay in which a secondary antibody is linked to an enzyme reporter group (ELISA). When exposed to its substrate, the enzyme will produce a product that can be detected by spectroscopic analysis or by another quantitative analytical method (e.g., a fluorescent, chemiluminescent, bioluminescent, phosphorescent or radiolabeled product). Suitable enzymes include, without limitation, alkaline phosphatase, glucose oxidase, beta-galactosidase, catalase, malate dehydrogenase, horseradish peroxidase, yeast alcohol dehydrogenase, and others.

Alternatively, the detection and quantitation of OPN in the fluid may be carried out using an antigen capture assay in which a subsaturating amount of unlabeled antibody (polyclonal, high affinity monoclonal or pooled monoclonal antibodies) is bound to the solid support, the sites for protein binding are blocked with a suitable blocking buffer (e.g., 3% BSA in PBS) and a fluid sample containing a fixed amount of labeled purified antigen (selected to provide sufficient signal within the linear range of binding to antibody) is added and allowed to bind to the antibody. After washing, the amount of labeled antigen is measured. The relative levels of antigen in different fluid samples can be determined, e.g., by assaying serial dilutions of each fluid sample and comparing the midpoints of the titration curves. The absolute amount of antigen in the sample can be determined by comparing the measured values with values obtained using known amounts of pure unlabeled antigen in a standard curve. The binding reaction between antibody and fluid sample containing labeled and unlabeled OPN is conveniently carried out in a microtiter plate. Alternatively, the binding may be carried out in solution and the complexes separated from the reaction mixture by contacting the reaction mixture with immobilized anti-immunoglobulin antibodies or proteins that are specific for an immunoglobulin, e.g., protein A or protein G.

An alternative method for detecting the level of an antigen in a fluid sample is to bind the fluid sample directly to a solid support, remove unbound proteins by washing, add an antibody specific for the antigen and allow it to bind. After removing unbound antibody by washing, the amount of antibody bound to the solid support is determined using a labeled secondary immunoreagent, e.g., a labeled anti-immunoglobulin antibody, protein A or protein G. This method is not useful if the antigen makes up a very small percentage of total proteins in the sample. For purposes of quantitation, the samples should contain similar amounts of proteins. Typically, solid supports with high protein binding capacity, e.g., nitrocellulose, are used, and both the primary and secondary antibodies are used in excess. Those of ordinary skill in the art using routine experimentation will be able to determine the optimal assay conditions required for detection of OPN in the samples.

Solid phase supports suitable for use in these assays will be known to those of ordinary skill in the art. These include microtiter wells, membranes, beads, magnetic beads, discs, gels, flat sheets, test strips, fibers and other configurations and types of materials that permit antigens and antibodies to be attached to the support. Attachment may be made by noncovalent or covalent means. Preferably, attachment will be made by adsorption of the antibody or antigen to a well in a microtiter plate or to a membrane such as nitrocellulose. These techniques are familiar to those skilled in immunology and are well known in the art.

To determine the presence or absence of tumor hypoxia in a patient according to the methods of this invention, the level of OPN, detected by methods such as those illustrated herein, is typically compared to a predetermined value that is capable of distinguishing between hypoxic tumors and oxic tumors in a specified patient population. The predetermined value may be an empirically determined value or range of values determined from test measurements on groups of patients with a particular class of tumor, e.g., head and neck, breast, or colon. Alternatively, the predetermined value may be based on values measured in a particular patient over a period of time. The Examples below illustrate methods by which a predetermined value for mean plasma OPN levels may be empirically determined in patients with squamous head and neck tumors. It should be understood by those of ordinary skill in the art that such methods are routine and can be used without undue experimentation with other classes of tumors and with fluids other than plasma, and are expected to be useful in human and non-human mammals.

In another preferred embodiment, the predetermined value is determined using a Receiver Operator Curve (described in ref. 28). This method may be used to arrive at the most accurate cut-off value, taking into account the false positive rate and the false negative rate of the diagnostic assay.

The assay can be performed in a flow-through or strip-test format by immobilizing the binding agent in a membrane. In a flow-through test, the sample is passed through the membrane and OPN contained in the sample complexes with the binding agent. A solution containing a second labeled binding agent is passed through the membrane and the amount of the detection reagent that binds to the complex is determined. In the strip test method, the membrane containing immobilized binding agent is dipped into a fluid sample from the patient. The sample migrates along the membrane through a region containing a second binding agent to the area containing immobilized binding agent. The amount of immobilized binding agent is selected to generate a visually detectable pattern when the sample contains a specified level of OPN. Antibodies and antigen-binding fragments are preferred for use in such assays, preferably in amounts ranging from 25 ng to about 1 ug, more preferably from about 50ng to about 500 ng. Very small amounts of patient samples are required for such a test. Examples of useful methods can be found in U.S. Pat. Nos. 5, 518,869 and 5,712,172.

The above descriptions are exemplary only, and are not intended to limit the scope of the invention in any way. It is recognized that those skilled in the art will know of other types of assays that are suitable for use in measuring OPN, its fragments and variants.

In another of its aspects, the present invention relates to a method of treating a patient with a malignant tumor by assessing the probability that the tumor is hypoxic and administering a hypoxia-selective tumor therapy, if warranted. Hypoxia-selective therapies (7) rely on the activation of certain anticancer drugs in the hypoxic tumor environment (e.g., bioreductive agents such as porfiromycin and mitomycin C), or on the use of hypoxic tumor sensitizers such as nitroimidazoles and tirapazamine in combination with other anticancer drugs or radiation therapy to achieve increased cytotoxic effects in tumors that contain hypoxic cells. Gene therapy approaches have also been proposed for use (7). Hypoxia response elements (HREs) can be linked to prodrug activating enzymes for gene therapy to selectively convert nontoxic prodrugs to toxic metabolites in solid tumors containing hypoxic cells. Genetically engineered anaerobic bacteria such as C. beijerinkii have been tested with some success for use in targeting hypoxic tumor cells.

In yet another aspect, the methods of the present invention are useful for monitoring the response of a patient's hypoxic tumor to therapy, and for predicting the risk of relapse following therapy. With the use of the noninvasive inventive methods described herein, OPN levels can be followed from the time that a hypoxic tumor is first diagnosed in a patient through various stages of therapy and following therapy to assess the likelihood of relapse. Furthermore, the pretreatment levels of OPN in patients with particular types of tumors are useful prognostic indicators in these patients.

The present invention also encompasses the use of OPN measurements in combination with measurements of other secreted hypoxia-induced proteins such as, for example, PAI-1, uPA, uPAR, TF, VEGF, adrenomedullin, transforming growth factor-alpha, and other hypoxia-induced gene products such as those described in PCT Application WO99/48916, for the diagnosis, prognosis, and therapy of cancer. In this regard, microarray technology is a convenient approach, although by no means the only approach that can be used.

In another aspect, the present invention encompasses screens for hypoxia-selective therapies based on measurement of OPN levels in bodily fluids of animal tumor models.

The present invention includes a kit for use in carrying out the methods of this invention comprising at least one binding agent (and optionally a detecting agent) for detecting a level of OPN in a fluid sample from a patient with cancer and a calibration means for comparing the level with a predetermined value or values

The aspects of the invention described herein are intended for use in human and veterinary medicine.

The following examples are presented solely to illustrate the practice of the invention, and not to limit the scope of the invention.

### EXAMPLE 1

### Linear Discriminant Analysis of NCI-60 Cancer Cell Line Gene Array Expression

The publicly available NCI-60 cancer cell line microarray expression database includes samples from nine different tissue types and was used as reported by Ross et al (23). The VHL and OPN genes were represented by two separate spots on each microarray. For each microarray, information from both spots was combined using the following formula: log₂ ratio=log₂ ({Spot 1 Chan1 Diff+Spot 2 Chan1 Diff}/{Spot 1 Chan2 Diff +Chan2 Diff}, where diff=difference in intensity of hybridized signals between the two spots. The cells were partitioned into two groups, high VHL expressers and low VHL expressers, based on VHL log₂ ratio. High VHL expressers were defined as those with VHL log₂ ratio>0.5 and the low expressers were those with ratio ≦−0.5.

Of the 60 cell lines in the NCI database, sixteen low VHL expressers and nine high VHL expressers were identified. The remaining cell lines had VHL log₂ ratios between 0.5 and −0.5 and were not considered further in this analysis. Using LDA, a machine learning algorithm (19,20) (see CLEAVer 1.0 at http://classify.stanford.edu for implementation of the algorithm), we were able to identify a gene profile that can best distinguish high VHL expressers from low expressers in these twenty-five cell lines with an estimated accuracy of 81.6% over ten trials. Within this gene profile, OPN was the highest weighted-gene, whose expression appeared to be inversely correlated with VHL expression (FIG. 1). Eighteen cell lines had adequate information on both OPN and VHL gene expression. Of these, eleven were low VHL expressers and seven were high expressers. Eight of eleven low VHL expressers had high OPN mRNA expression on the gene array.

Northern blot analysis confirmed the inverse relationship of VHL and OPN gene expression (data not shown). Cells that expressed intermediate to high levels of OPN mRNA had low levels of VHL mRNA. Cells that expressed undetectable levels of OPN mRNA expressed elevated levels of VHL mRNA. ELISA analysis of OPN protein levels in these cell lines validated the Northern blot results (data not shown).

### EXAMPLE 2

### Comparison of Plasma OPN Levels in VHL Patients and Normal Volunteers

The VHL patients consisted of thirty-one patients with a confirmed VHL diagnosis by genetic screening. The control group consisted of 15 healthy volunteers (7 males and 8 females) who were in a similar age group as the VHL patients. All study subjects signed an IRB approved informed consent form.

A five ml sample of blood was obtained by venipuncture into a vacutainer coated with 3.2% sodium citrate buffer as anticoagulant. The samples were centrifuged at 3000 rpm at 4° C. for 10 minutes within 30 minutes of collection. The separated plasma was removed, aliquoted, and stored at −80° C. prior to analysis.

Plasma levels of OPN protein were measured using an ELISA method (Assay Designs, Inc, Ann Arbor, Mich.) according to the instructions of the manufacturer. The OPN ELISA consists of OPN polyclonal rabbit antibodies immobilized on a microtiter plate to bind OPN in the sample. The ELISA is designed to detect human OPN in biological fluids with a detection limit >2.2 ng/ml, a 5% intra-assay and a 2% inter-assay variability, and an 88% recovery rate from human EDTA plasma.

The t-test was used to compare the difference in the mean OPN levels between VHL patients and healthy volunteers.

As shown in FIG. 2, OPN plasma levels were elevated in patients lacking the VHL tumor suppressor gene, compared with healthy volunteers. The mean OPN level for healthy volunteers was 318 ng/ml (range: 233-461) and that for VHL patients was 447 ng/ml (range: 261-843.2). The difference of OPN level between the two groups was statistically significant by the t- test (p=0.002).

### EXAMPLE 3

### OPN Levels in Plasma of Patients with Oxic and Hypoxic Tumors

The patient population in this study consisted of fifty-four adults evaluated at the Stanford Head and Neck Cancer Tumor Board with newly diagnosed, histologically confirmed, head and neck squamous cell carcinoma (HNSCC) without prior radiation or chemotherapy treatment. All patients admitted to the study had either a primary tumor or a regional lymph node that was easily accessible to pO₂ measurements with the Eppendorf polarographic microelectrode. All participating patients signed an informed consent approved by the Institutional Review Board and the US Department of Health and Human Services. Forty-nine of the patients in the study had both OPN and pO₂ measurements while five patients had OPN measurements only. Table 3 shows patient, tumor and treatment characteristics.

TABLE 3

| | |
|---|---|
| Characteristic | No. of patients (%) |

| | | | |
|---|---|---|---|
| Age | ≦55 | 29 | (54) |
| | >55 | 25 | (46) |
| Gender | Male | 40 | (74) |
| | Female | 14 | (26) |
| Tumor site | Oral cavity | 12 | (22) |
| | Oropharynx | 30 | (56) |
| | Hypopharynx | 8 | (15) |
| | Others | 4 | (7) |
| Tumor volume | ≦28 cm³ | 23 | (43) |
| | >28 cm³ | 24 | (44) |
| | Unknown | 7 | (13) |
| Median Tumor pO₂ | ≦10 mm Hg | 24 | (44) |
| | >10 mm Hg | 25 | (46) |
| | Unknown | 5 | (9) |
| Hemoglobin | <14 g/dL | 25 | (46) |
| | ≧14 g/dL | 28 | (52) |
| | Unknown | 1 | (2) |
| OPN level | ≦450 ng/ml | 29 | (54) |
| | >450 ng/ml | 25 | (46) |
| Surgery | No | 28 | (52) |
| | Yes | 26 | (48) |
| Chemotherapy | No | 14 | (26) |
| | Yes | 40 | (74) |
| Tirazapazamine | No | 37 | (69) |
| | Yes | 17 | (31) |
| Radiotherapy | No | 4 | (7) |
| | Yes | 50 | (93) |

The staging evaluation for all patients included history and physical examination, panendoscopy and examination under anesthesia, chest radiographs, complete blood count and liver function tests. All patients also had head and neck imaging studies, either computed tomography (CT) or magnetic resonance imaging (MRI). All were staged according to the 1988 AJCC staging system (30). The TNM staging distribution of the patients in this study is shown in Table 4.

TABLE 4
TNM staging distribution of 54 patients

| | T-stage | | | | | |
|---|---|---|---|---|---|---|
| N-stage | 0 | 1 | 2 | 3 | 4 | Total |
| 0 | 0 | 0 | 3 | 0 | 5 | 8 |
| 1 | 0 | 0 | 0 | 0 | 1 | 1 |
| 2 | 2 | 2 | 12 | 7 | 11 | 34 |
| 3 | 1 | 4 | 3 | 0 | 3 | 11 |
| Total | 3 | 6 | 18 | 7 | 20 | 54 |

All oxygen tension measurements were performed using a computerized histograph (Sigma Eppendorf pO₂ Histograph, Kimoc 6650, Hamburg, Germany (25). The tissue oxygen partial pressure (pO2) was measured polargraphically using a fine-needle oxygen electrode, which was inserted either directly into the tumor or subcutaneously through a 22-gauge intravenous catheter. Machine calibrations with room air and 100% nitrogen were performed before and after each series of Eppendorf measurements. Fifty to eighty pO₂ measurements in 2-3 separate tracks were recorded from each tumor and an equal number of pO₂ measurements were taken from normal adjacent subcutaneous tissues. In all patients, the median tumor pO₂ was consistently lower than that of normal subcutaneous tissues from the same patient. Measurements of tumor pO₂ were made at the primary tumor site in eight patients and from pathologically involved neck nodes in 41 patients. The measurements were presented in the form of histograms, along with the calculation of a median pO², % of values<2.5 mm Hg (HF2.5) and percent of values<5 mm Hg (HF5) for each measured site.

FIG. 4 shows the correlation between the median pO₂ and plasma OPN levels in this patient group with a correlation coefficient of −0.42 and a p-value of 0.003 using the Spearman rank method. Although various factors may adversely influence the correlation between the two methods, there is nevertheless a significant correlation between OPN and tumor pO₂, which suggests the utility of OPN as a serum marker for identifying patients with hypoxic tumors.

### EXAMPLE 4

### Statistical Methods

Statistical analysis was performed using Statistix (Analytical Software Inc, Tallahassee, Mich.) and Stata statistical software. (Stata Corp., College Station, Tex.). Stata was purchased from Computing Resource Center, Inc., Santa Monica, Calif.). The Spearman rank test was used to determine the relationship between OPN and median tumor pO₂. The stepwise linear regression method was used in multivariate analysis to identify factors that correlated with median tumor pO₂ (24). Studied variables included patient age, gender, pack-year of cigarettes used, tumor stage, nodal stage, hemoglobin (Hb) and OPN levels. Freedom from relapse (FFR) was computed with the Kaplan-Meier product-limit method (29). Outcomes were measured from the date of diagnosis to the date of any failure (including local, regional or distant failures). Log-rank statistics were employed to identify important prognostic factors for FFR and overall survival. Analyzed variables included age, gender, tumor stage, nodal stage, tumor volume, treatment methods, median tumor pO₂, Hb and OPN levels. Only those factors that achieved statistical significance on univariate analysis (i.e., p value<0.05) were entered into a step-wise Cox proportional hazard model for multivariate analysis (31). The t- test was used to compare the difference in the mean OPN levels between VHL patients and healthy volunteers (24).

### EXAMPLE 5

### OPN Expression in Normoxic and Hypoxic Tumor Cells

Human SCC 4 cells (tongue squamous cell carcinoma line; ATCC, Rockville, Md.) were cultured in media containing 10% (v/v) fetal calf serum as specified by ATCC. Just prior to treatment, the cells were washed with PBS and incubated in serum-free media. Hypoxia was induced by incubating cells in a 37° incubator (Sheldon Manufacturing Inc.) which maintained an environment of less than 0.05% oxygen. Control (normoxic) cells were treated similarly but were maintained in a 37° C. incubator in a 5% CO₂/95% O2 environment. All experiments were performed at 70-80% cell confluence in media with a pH of 7-7.4 for the duration of the experiment.

Levels of intracellular and secreted OPN were measured in cultures exposed to hypoxic conditions for 2 hours, 4 hours, 6 hours, 12 hours and 24 hours, and in cultures exposed to normoxic conditions for 24 hours. For intracellular measurements, cells were lysed with standard lysis buffer (RIPA buffer containing protease inhibitors). OPN was measured by ELISA assay as described above in Example 2. The results shown in FIG. 3 show the average and standard deviation for duplicate samples. Values of OPN were normalized for protein concentration (measured by BCA method) in the samples analyzed. OPN levels in the media of cells exposed to hypoxic conditions were 0 ng/ml at 2 hours and 4 hours, 1.2 ng/ml at 6 hours, 123 ng/ml at 12 hours and 322 ng/ml at 24 hours. Normoxic cells secreted only a low level of OPN into the medium after 24 hours, which was less than the level secreted by cells exposed to hypoxic conditions for the same period of time. The levels of OPN in the lysates of hypoxic cells peaked at 12 hours and by 24 hours were comparable to OPN levels in normoxic cell lysates. These results suggest that hypoxia may regulate OPN secretion by tumor cells.

### REFERENCES

1. Denhardt, D. T and Guo, X. “Osteopontin: a protein with diverse functions”. FASEB J. 7: 1475-82 (1993).

2. Singhal, H. et al, “Elevated plasma osteopontin in metastatic breast cancer associated with increased tumor burden and decreased survival,” Clin. Cancer Res. 3: 605-11 (1997).

3. Devoll, R. E.- et al, “Osteopontin(OPN) distribution in premalignant and malignant lesions of oral epithelium and expression in cell lines derived from squamous cell carcinoma of the oral cavity,” J. Oral Pathol Med. 28: 97-101 (1999).

4. Ue, T. et al, “Co-expression of osteopontin and CD44v9 in gastric cancer,” Int. J.

Cancer 79: 127-32 (1998).

5. Saitoh, Y. et al, “Expression of osteopontin in human glioma. Its correlation with the malignancy,” Lab. Invest. 72: 55-63 (1995).

6. Thomlinson, R. H. and Gray, L. H., “The histological structure of some human lung cancers and the possible implications for radiotherapy,” Br. J. Cancer 9: 539-549 (1955).

7. Brown, J. M. and Giaccia, A. J., “The unique physiology of solid tumors: opportunities (and problems) for cancer therapy,” Cancer Res. 58: 1408-1416 (1998).

8. Koong, A. C. et al, Cancer Res. 60: 883-887 (2000).

9. PCT Application WO99/48916.

10. Brizel, D. M. et al, “Tumor oxygenation predicts for likelihood of distant metastasis in human soft tissue sarcoma,” Cancer Res. 56: 941-943 (1996).

11. Hockel, M. et al, “Association between tumor hypoxia and malignant progression in advanced cancer of the uterine cervix,” Cancer Res. 56: 4509-4515 (1996).

12. Fyles, A. W. et al, “Oxygenation predicts radiation response and survival in patients with cervix cancer,” Radiother. Oncol. 48: 149-156 (1998).

13. Nordsmark, M. et al, “Pretreatment oxygenation predicts radiation response in advanced squamous cell carcinoma of the head and neck,” Radiother. Oncol. 41: 31-39 (1996).

14. Brizel, D. M. et al, “Oxygenation of head and neck cancer: changes during radiotherapy and impact on treatment outcome,” Int. J. Radiat. Oncol. Biol. Phys. 42: 147 (1998).

15. Stone, H. B. et al, “Oxygen in human tumors: correlations between methods of measurement and response to therapy,” Radiat. Res. 136: 422-434 (1993).

16. Zbar, B. et al, “Third International Meeting on von Hippel-Lindau disease,” Cancer Res. 59: 2251-3 (1999).

17. Cockman, M. E. et al, “Hypoxia inducible factor-alpha binding and ubiquitination by the von Hippel-Lindau tumor suppressor protein,” J. Biol. Chem. 275: 25733-41 (2000).

18. Wykoff, C. C et al, “Identification of novel hypoxia dependent and independent target genes of the von Hippel-Lindau (VHL) tumour suppressor by mRNA differential expression profiling,” Oncogene 19: 6297-6305 (2000).

19. Riley, B. D., in “Pattern Recognition and Neural Networks,” p. 91-120, Cambridge University Press, 1999.

20. Raychaudhury, S. et al, “Basic microarray analysis: grouping and feature reduction,” Trends in Biotechnology 19: 189-193 (2001)

21. Denko, N. et al, “Epigenetic regulation of gene expression in cervical cancer cells by the tumor microenvironment”, Clin. Cancer Res. 6: 480-7 (2000).

22. Denhardt, D. T. et al (Eds), “Osteopontin: Role in Cell Signalling and Adhesion,” Ann. NY Acad Sci. 760 (1995).

23. Ross, D. T. et al, “Systemic variation in gene expression patterns in human cancer cell lines”, Nat. Genet. 24: 227-235 (2000).

24. Glanz, S. A. and Slinker, B. K. “Primer of applied regression analysis of variance,” pp. 50-109, 512-168 (McGraw-Hill, Inc., New York, 1990.

25. Terris, D. J. and Dunphy, E. P., “Oxygen tension measurements of head and neck cancers,” Arch. Otolaryngol. Head Neck Surg. 120: 283-287 (1994).

26. Harlow and Lane, “Antibodies: A Laboratory Manual,” Cold Spring Harbor Laboratory, 1988.

27. Kohler and Milstein, Continuous cultures of fused cells secreting antibody of predefined specificity, Nature 256: 495-497 (1975).

28. Sackett et al, “Clinical Epidemiology: A Basic Science for Clinical Medicine,” pp. 106-7, Little Brown and Co., 1985.

29. Kaplan, E. S. and Meier, P., “Non-parametric estimations from incomplete observations,” Am. Stat. Assoc. J. 53: 457-482 (1958).

30. Sobin, L. H. et al, “TNM classification of malignant tumors. A comparison between the new (1987) and the old editions,” Cancer 61: 2310-2314 (1988).

31. Cox, D. R., “Regression models and life tables,” J R Stat Soc 34: 187-220 (1972).

32. Kim, J. H. et al, “Osteopontin as a potential diagnostic biomarker for ovarian cancer,” JAMA 287(13): 1671-1679 (2002).

33. Agrawal D. et al, “Osteopontin identified as lead marker of colon cancer progression, using pooled sample expression profiling,” J. Natl Cancer Inst 94(7): 513-521 (2002).

The above references are cited in this application. All of the publications, patent applications and patents cited in this application are herein incorporated by reference in their entirety to the same extent as if each individual publication, patent application or patent was specifically and individually indicated to be incorporated by reference in its entirety.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, to the objective spirit and scope of the present invention. All such modifications are intended to be within the scope of the claims appended hereto.

What is claimed is:

1. A method for diagnosing tumor hypoxia in a patient with cancer, comprising detecting a level of osteopontin (OPN) in a bodily fluid of the patient and comparing the level with a predetermined value or values.

2. The method of claim 1, wherein said level is detected by contacting said bodily fluid with a binding agent specific for OPN and measuring the amount of the agent that is specifically bound in a complex with OPN.

3. The method of claim 2, wherein the binding agent comprises at least one antibody.

4. The method of claim 2, further including a fixed amount of labeled OPN, wherein said OPN in a bodily fluid and said added labeled OPN compete for binding to said binding agent.

5. The method of claim 2, wherein the binding agent is bound to a solid support.

6. The method of claim 2, wherein the binding agent comprises a reporter group.

7. The method of claim 2, wherein the amount of the binding agent specifically bound in a complex with OPN is determined with a detecting reagent comprising a reporter group.

8. The method of claim 2, wherein the level of OPN is detected by an ELISA assay.

9. The method of claim 1, wherein said bodily fluid is blood.

10. The method of claim 1, wherein the predetermined value or values is empirically determined in a group of cancer patients.

11. The method of claim 1, wherein the predetermined value or values is obtained from the patient being diagnosed for tumor hypoxia.

12. A method for treating a patient with a malignant tumor, comprising diagnosing whether the tumor is hypoxic according to the method of claim 1, and administering a therapy that includes a hypoxia-selective tumor therapy for a tumor that is diagnosed as hypoxic.

13. A method for modulating the response of a tumor to radiation or chemotherapy, comprising diagnosing the presence of hypoxia according to the method of claim 1, and administering a hypoxic tumor sensitizer combined with the radiation or chemotherapy treatment.

14. A method for monitoring the response of a patient's hypoxic tumor to therapy, comprising determining the level of OPN in a bodily fluid obtained from the patient prior to therapy and at various times during and after therapy, and comparing the level to a predetermined value or values that are indicative of the presence of tumor hypoxia.

15. The method of claim 1, wherein the cancer is squamous cell carcinoma of the head and neck.

16. The method of claim 12, wherein the cancer is squamous cell carcinoma of the head and neck.

17. The method of claim 13, wherein the cancer is squamous cell carcinoma of the head and neck.

18. The method of claim 14, wherein the cancer is squamous cell carcinoma of the head and neck.

19. A kit for detecting the presence of a hypoxic tumor in a cancer patient comprising a binding agent for determining a level of OPN in a fluid sample from the patient and a calibration means for comparing the level with a predetermined value or values.

20. The method of claim 1, further comprising detecting at least one other secreted hypoxia-induced protein in a bodily fluid of the patient.

21. A method for assessing the prognosis of a patient with squamous cell carcinoma of the head and neck comprising determining the plasma OPN level in said patient prior to treatment and comparing the level to predetermined values for OPN levels that are predictive of freedom-from-relapse rates and survival rates in a patient population with squamous cell carcinoma of the head and neck.
